(19) United States
(12) Patent Application Publication
(10) Pub. No.: US 2026/0191685 A1
(43) Pub. Date: Jul. 9, 2026

(54) Myopia Control Device and Methods of Use

(71) Applicant: Michael James Berry, Austin, TX (US)

(72) Inventor: Michael James Berry, Austin, TX (US)

(73) Assignee: VIS, Inc., Austin, TX (US)

(21) Appl. No.: 18/831,986

(22) Filed: Dec. 29, 2025

Related U.S. Application Data
(60) Provisional application No. 63/833,710, filed on Jan. 7, 2025.

(51) Int. Cl. A61F 9/008

(52) U.S. Cl. CPC A61F 9/008; A61F 2009/00872; A61F 2009/00878; A61F 2009/00885

(57) ABSTRACT

The present invention includes a myopia control ophthalmic medical device (incorporating, but not limited to, one or more lasers, an optical fiber array for delivery of laser light to a sapphire applanation window/suction ring that is mounted on the eye) and methods for use of the device (including, but not limited to, use of specific laser parameters and treatment patterns) to personalized treatment of the eye for myopia control (i.e., reduction of myopia, myopic astigmatism, age-related myopia increase and age-related myopic astigmatism increase) to improve, stabilize or restore vision. The applications of the present invention include, but are not limited to, myopia control. The myopia control devices and methods of the present invention can also be used in combination with other therapies including, but not limited to, pharmacological therapies.

### CROSS-REFERENCE

### U.S. Provisional Patent Application

US 63/833,710 filed 17 Jan. 2025

### BRIEF SUMMARY OF THE INVENTION

This invention is a myopia control ophthalmic medical device and methods for its use to provide myopia control (e.g., control of nearsightedness and its age-dependent increase in children).

### Key Features Include

A myopia control ophthalmic medical device comprising: 1) an adjustable medical laser that can deliver predetermined light energy to the cornea, 2) an optical fiber array to deliver light energy and 3) a sapphire applanation window/suction ring (SAWSR) accessory that permits accurate delivery of light energy to the cornea without causing clinically significant corneal epithelial damage.
Methods for use of the myopia control ophthalmic medical device to provide required (personalized) treatment of patient eyes.

### DESCRIPTION OF THE DRAWINGS

For a more complete understanding of this disclosure and its features, reference is made to the following description, taken in conjunction with the accompanying drawings, in which:

FIG. 1 illustrates an example system for corneal reshaping according to this disclosure.

FIG. 2 shows example treatment patterns suitable for myopia control. Each treatment pattern is displayed on a grid of concentric diameters at 1 mm intervals. Detailed information on each example treatment pattern is presented in paragraph [00020].

### FIELD OF THE INVENTION

The present ophthalmic invention includes a myopia control ophthalmic medical device and methods for its use (including, but not limited to, specific treatment patterns) to provide myopia control (i.e., reduction of myopia, myopic astigmatism and/or reduction of the age-related increase of myopia and myopic astigmatism) to improve, stabilize or restore vision. The applications of the present invention include, but are not limited to, myopia control. The myopia control devices and methods of the present invention can also be used in combination with other therapies including, but not limited to, pharmacological therapies.

### BACKGROUND

Myopia (nearsightedness) is the most prevalent refractive error globally, with an estimated prevalence of 3 billion people in 2025 and a projected prevalence of nearly 5 billion people in 2050 [Ref. 1]. Of particular importance is the increasing prevalence of age-related increase of myopia in children, which has reached epidemic proportions [Ref. 2]. Age-related myopia increase may advance to pathological conditions including, but not limited to, retinal detachment and myopic maculopathy. The rate of age-related myopia increase depends upon baseline refraction, with children having hyperopia reserve (HR) having less rapid increase [Ref. 3].

Myopia control (e.g., reduction of age-related myopia increase) can be achieved, at least in part, by current approaches such as spending time outdoors in sunlight and by use of devices including, but not limited to, specialized spectacles, specialized multifocal contact lenses, orthokeratology lenses and surgical lasers. Pharmacological approaches including, but not limited to, atropine administration to the eye are also used to reduce the rate of age-related myopia increae. These current approaches have limitations including safety, efficacy, side effects, convenience and cost. These limitations are overcome, at least in part, by the present invention.

### SUMMARY OF THE INVENTION

The invention described herein includes myopia control (e.g., reduction of age-related myopia increase) devices and methods to optically reshape the cornea of the eye in order to modify the refractive status of the eye.

### DETAILED DESCRIPTION OF THE INVENTION

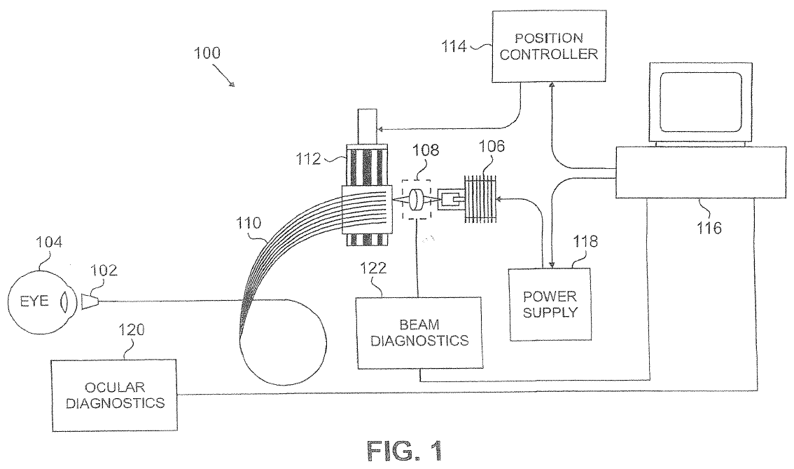

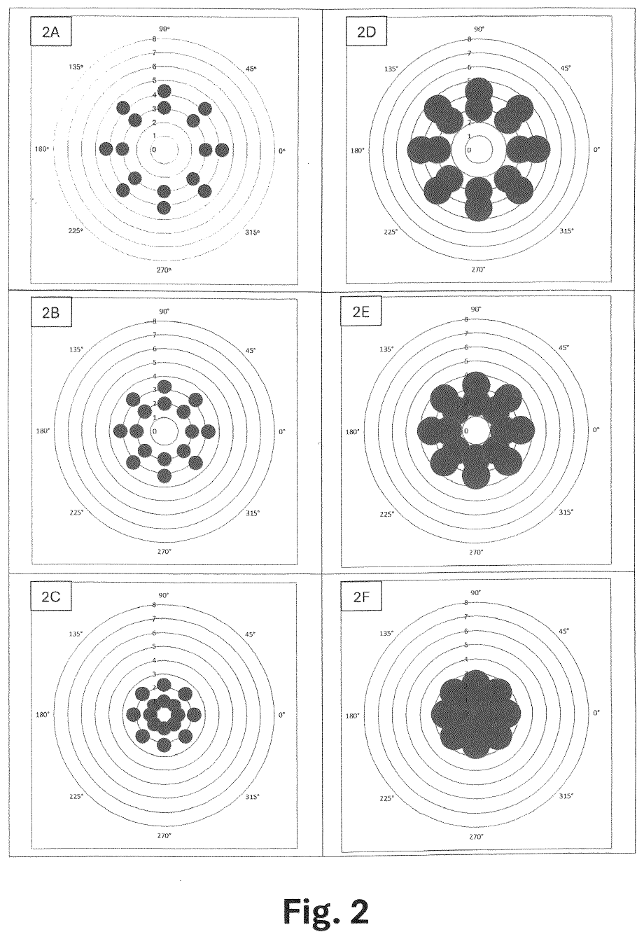

FIGS. 1 through 2, described below, and the various embodiments used to describe the principles of the present invention in this patent document are by way of illustration only and should not be construed in any way to limit the scope of the invention. Those skilled in the art of invention will understand that the principles of the invention may be implemented in any type of suitably arranged device or system.

The device is essentially the same as that described previously [Ref. 4].FIG. 1 illustrates an example device 100.for cornea reshaping according to this disclosure. The embodiment of the device shown in FIG. 1 is for illustration only. Other embodiments of the device 100 may be used without departing from the scope of this disclosure.

In this example, device 100 includes a sapphire applanation window/suction ring (SAWSR) 102 that is mounted on the patient eye 104 during a corneal reshaping procedure. SAWSR 102 includes a sapphire applanation window that is in contact with the cornea to provide epithelial protection from thermal damage during laser irradiation [Ref. 4].

In this example, device 100 includes laser 106 that produces light that is directed through optical fiber array 110 to deliver laser light through SAWSR 102 to eye 104. Laser 106 parameters including, but not limited to, wavelength, energy and duration are characterized using beam diagnostics 108. Laser 106 is powered by power supply 118. Optical fiber array 110 is positioned using translation stage 112 that is operated using position controller 114. Translation stage 112 may be operated with one or more lasers for sequential or simultaneous delivery of laser light through one or more fibers in optical fiber array 110. Ocular characteristics are measured by ocular diagnostics 120 and that information is used by computer 116 to calculate predetermined laser parameters including, but not limited to, laser energy output and pulse duration. Computer 116 operates laser 106 with the predetermined parameters.

Laser 106 may be one or more lasers including, but not limited to, thulium fiber lasers (TFLs) or semiconductor diode lasers (SDLs). Laser 106 produces light with predetermined laser parameters including, but not limited to, wavelength, energy and duration.

Laser 106 light is delivered sequentially through one or more fibers in optical fiber array 110 or simultaneously through multiple fibers in optical fiber array 110. In sequential operation, laser 106 light from a single laser is delivered through one or more fibers in optical fiber array 110 using translation stage 112 and position controller 114 to couple individual fibers with laser 106 light to deliver each laser treatment (Tx) spot sequentially through SAWSR 102 to eye 104. In simultaneous operation, laser 106 light from one or more lasers is delivered through two or more fibers in optical fiber array 110 using translation stage 112 and position controller 114 to couple one or more fibers with laser 106 light to deliver laser Tx spots simultaneously through SAWSR 102 to eye 104. In simultaneous operation, laser 106 light from one or more lasers is used to produce 2 or more beams that are delivered through two or more fibers in optical fiber array 110 using translation stage 112 and position controller 114 to couple two or more fibers with laser 106 light to deliver laser Tx spots simultaneously through SAWSR 102 to eye 104. Two or more beams of laser light may be derived from one or more lasers using one or more axicon lenses or beamsplitter and mirror arrays.

SAWSR 102 is mounted on the patient eye 104 to be treated using suction. SAWSR 102 includes, but is not limited to, a sapphire applanation window (SAW) mounted within a suction ring (SR) that is equipped with a vacuum port that is connected to a suction source. SAWSR 102 may include, but is not limited to, one or more accessories such as a reticle on the SAW and a light source on the SR, both of which aid in mounting SAWSR 102 on eye 104 accurately with respect to a predetermined position reference such as the pupil center of eye 104.

### Preferred Embodiments of the Invention

In type L1 laser embodiments of the invention, laser 106 is one or more thulium fiber lasers (TFLs) operated in continuous wave (cw) mode. In type LIA laser embodiment, laser 106 light pulse durations are produced by one or more optical shutters. For sequential delivery of laser light pulses, shutter open/off times are synchronized with translation stage 112 positions to couple fibers of optical fiber array 110 with laser 106 light pulses to deliver each laser treatment spot (from one TFL) or two or more laser treatment spots (from two or more TFLs) sequentially through SAWSR 102 to eye 104. In type L1B laser embodiment, laser 106 light pulse durations are produced by one or more optical shutters. For simultaneous delivery of laser light pulses, shutter open/off times are synchronized with translation stage 112 positions to couple one or more fibers of optical fiber array 110 with laser 106 light pulses to deliver one or more laser treatment spots simultaneously from one or more TFLs through SAWSR 102 to eye 104.

In type L2 laser embodiments of the invention, laser 106 is a set of one or more semiconductor diode lasers (SDLs) operated in repetitively pulsed (rp) mode. For sequential delivery of laser light pulses from one or more SDLs, shutter open/off times are synchronized with translation stage 112 positions to couple individual fibers of optical fiber array 110 with laser 106 light pulses to deliver each laser Tx spot sequentially through SAWSR 102 to eye 104.

In both L1 and L2 laser embodiments of the invention, laser 106 produces light in one or more wavelengths that correspond to cornea absorption coefficients in the range of 5 to 500 cm⁻¹. An example of these wavelengths is in the 2 μm spectral region within the 1.75 to 2.25 μm corneal absorption band. An example is a laser 106 that produces a wavelength of 1.93 μm [Ref. 4].

In both L1 and L2 laser embodiments of the invention, laser 106 produces one or more pulses of light with pulse durations in the range of 5 to 500 ms. An example is a laser 106 that produces 4 sets of pulses with pulse durations of 150 ms [Ref. 4].

In both L1 and L2 laser embodiments of the invention, laser 106 produces one or more treatment (Tx) spot energies that irradiate eye 104 in the range of 5 to 500 mJ per spot. An example is a laser 106 that produces Tx spot energies of 20 to 70 mJ per spot [Ref. 4].

In optical fiber array embodiments of the invention, optical fiber array 110 includes one or more optical fibers that transmit laser light and that are arranged in treatment (Tx) patterns. FIG. 2 shows example Tx patterns that are useful for myopia control. These patterns include 16 Tx spots of 0.5 mm diameter in FIGS. 2A, 2B and 2C arranged symmetrically with 8 spots on 3.0/4.2 mm (FIGS. 2A), 2.0/3.2 mm (FIGS. 2B) and 1.0/2.2 mm (FIG. 2C) centerline diameters of the inner/outer rings. These patterns also include 16 Tx spots of 1.0 mm diameter in FIGS. 2D, 2E and 2F arranged symmetrically with 8 spots on the same centerline diameters of inner/outer rings as FIGS. 2A, 2B and 2C, respectively. The Tx spots can be variable in number from 1 through 16 or greater, circular or non-circular, equal or unequal in spot diameter or non-circular shape, equal or unequal in location (both distance and angular orientation) with respect to a centration reference, and equal or unequal in Tx spot energy. The Tx spots can also be configured to have predetermined distributions of laser energy within each spot. These variables are adjusted (personalized) as required for a predetermined reduction of myopia and/or myopic astigmatism in each eye. An example for hyperopia and presbyopia treatment (Tx), but not myopia Tx, is a Tx pattern of 16 spots of 0.5 mm diameter arranged symmetrically with 8 spots on 6.0/7.2 mm centerline diameters of the inner/outer rings [Ref. 4]. Tx patterns of Tx spots at large centerline diameters (such as 6.0/7.2 mm) produce multifocal corneas with central steepening as demonstrated previously [Refs. 4, 5 and 6] while Tx spots at small centerline diameters (such as 3.0/4.2 mm) produce corneas with central flattening as demonstrated in animal experiments [Ref. 5] and in human eyes [Ref. 6]. The fiber optic array 110 has a distal handpiece that contains accurately positioned fibers and that is docked accurately and securely onto the SAWSR 102 using matching handpiece and SAWSR magnets [Ref. 4].

In sapphire applanation window/suction ring (SAWSR) embodiments of this invention, SAWSR 102 includes a sapphire or non-sapphire applanation window that transmits laser light to the eye 104 and that has example dimensions of, but not limited to, 10.0 mm diameter and 0.1 mm thickness.

While the description of the invention herein shows, describes, and points out novel features as applied to various embodiments, it will be understood that various omissions, substitutions, and changes in the form and details of the device or method illustrated can be made without departing from the spirit of the disclosure. As will be recognized, certain embodiments of the inventions described herein can be embodied within a form that does not provide all of the features and benefits set forth herein, as some features can be used or practiced separately from others. All changes that come within the meaning and range of equivalency of the claims are to be embraced within their scope.

This invention can be used in combination therapy including, but not limited to, medications such as atropine, neurotransmitters such as dopamine that decrease excessive axial elongation of the eye, good environmental measures such as increased exposure to sunlight, or any other therapy that reduces excessive axial elongation of the eye during childhood and later in life.

### Benefits and Advantages of the Invention

A previous laser 106 and its use to treat human eyes was unsatisfactory for myopia control since it produced highly damaged Tx spots that were prominently visible due to their dense (high) opacity, depth (greater than 200 μm) and long persistence (one month or longer post-Tx) in a Tx pattern that would be suitable for corneal flattening [Ref. 6]. Those unsatisfactory Tx spots at 3.0 mm centerline diameter were within the central region of the cornea and typically within the pupil diameter, thereby raising concern about their effect on visual acuity and the quality of vision. The laser 106 and its use to treat human eyes described herein in this invention produces undamaged Tx spots that are barely visible due to their light (low) opacity. depth (less than ca. 150 μm) and short persistence (typically fading significantly within a few days post-Tx) [Ref. 4]. The Tx spots of this invention are in the central region of the cornea but are out-of-focus and hence not observed by any patient whose eye(s) are treated. The Tx spots of this invention produce little or no light scattering so corneal clarity (without significant opacity and possible dysphotopsias such as glare, starburst and halo) is conserved. The Tx spots of this invention are highly suitable for myopia control.

The procedure for use of this invention is safe, repeatable, noninvasive and simple and rapid for both patient and physician [Ref. 4]. Patient comfort is excellent during treatment and there are typically only minor discomforts (such as, but not limited to, photophobia and foreign body sensation) for a few days post-treatment.

There is little or no post-treatment recovery period or need for aftercare.

Patients of all ages with and/or at risk of myopia, myopic astigmatism and age-related increase of these ametropias can be treated using this invention for temporary reduction of their refractive errors. Symmetrical corneal flattening and hence myopia and regular myopic astigmatism reduction is extended in duration by preselection of laser 106 parameters and Tx patterns. Irregular myopic astigmatism can also be reduced by preselection of specialized patterns. This invention offers a practical alternative to other methods of myopia control such as surgery or contact lenses.

Children and adolescents (typically in the 4-to 18-year-old age range) with, or at risk of, myopia, high myopia and age-related myopia increase can be treated using this invention. Surgery (using excimer and/or femtosecond lasers) is not authorized by FDA and other regulatory agencies for patients less than 18 years old. The main current treatment of these children is with the use of low dose (0.01% to 0.05%) atropine in aqueous solution that is administered in eye drops daily [Ref. 3]. Atropine medication has side effects such as photophobia and reduced accommodation that affect near vision. Low dose atropine medication is only partly effective-for example, in one study for children of ages 4 to 9 without baseline myopia, children with fast myopic shift (spherical equivalent change of 0.5 D or less over 12 months or 1.0 D or less over 24 months) remained at 25.0% prevalence for 0.05% atropine [Ref. 7]. While this outcome was better than that for lower dose atropine (for example, children with fast myopic shift remained at 45.1% prevalence for 0.01% atropine), these outcomes are not much better than placebo with 53.9% prevalence. The invention described herein is likely to reduce fast myopic shift better than atropine and/or other modalities presently used.

An even more promising use of this invention is for prevention or reduction of myopia and age-related myopia increase in children by producing and/or conserving hyperopia reserve (HR). If children are treated at an early age to give them, or conserve, more than 1.0 D of HR, they may experience little or no myopic shift as they age [Ref. 3]. Any myopic shift that remains can be reduced or eliminated by repeated treatments using this invention.

### REFERENCES

1—Holden B A, et al. Global prevalence of myopia and high myopia and temporal trends from 2000 to 2050. Ophthalmology 2016; 123:1036-1042.
2—Morgan I G, et al. The epidemics of myopia: aetiology and prevention. Prog Retin Eye Res 2018; 62:134-149.
3—Wang J, et al. Normative value of hyperopia reserve and myopic shift in Chinese children and adolescents aged 3-16 years. Br J Ophthalmol 2024; 108:1024-1029.
4—Rodgers K J, et al. Improved method of laser thermal keratoplasty to overcome presbyopia. In Ophthalmic Technologies XXI, edited by Soderberg M F, et al., Proc SPIE 2011; 7885: N−1 through N−8.
5—Morerira H, et al. Holmium laser thermokeratoplasty. Ophthalmology 1993; 100:752-761.
6—Ariyasu R G, et al. Holmium laser thermal keratoplasty of 10 poorly sighted eyes. J Refract Surg 1995; 11:358-3657.
7—Yam J C, et al. Ten-year change in visual function and incidence of visual impairment in highly myopic children and adults. JAMA 2023; 329:472-481.

1. A myopia control device
Wherein the device comprises one or more laser sources of light configured to produce one or more predetermined wavelengths of light, one or more predetermined durations of light and one or more predetermined energies of light to cause irradiation of the cornea.

2. The device of claim 1 that includes one or more of the following accessories:
2A—A fiber optic delivery accessory containing one or more optical fibers that deliver one or more beams of light onto one or more specific areas on the cornea,
2B—A suction ring accessory that mounts onto the cornea with applied vacuum from a vacuum pump and that allows the fiber optic delivery accessory to be docked onto the suction ring accessory. and
2C—A thermally conductive optical window accessory on the suction ring accessory that is configured to be in contact with the surface of the corneal epithelium and that is configured to provide cooling of the corneal epithelium to protect it from thermal damage.

3. The device of claim 2 in which predetermined laser treatment conditions of light delivered to one or more areas of the cornea include;
3A—One or more wavelengths of light irradiating one more treated areas that correspond to corneal absorption coefficients in the range of 5 to 500cm⁻¹,
3B—One or more patterns of light delivered to the cornea in one or more treated areas,
3C—One or more durations of light delivered to the cornea in the range of 5 to 500 ms in one or more treatment areas, and
3D—One or more energies of light delivered to the cornea in the range of 5 to 500 mJ per treatment area in one or more areas of the cornea.

4. The device of claim 3 in which one or more myopia control treatments are personalized to treat one or more of the following conditions: myopia, myopic astigmatism, and age-dependent myopia increase and/or myopic astigmatism increase.

5. The device of claim 4 in which predetermined laser treatment conditions are adjusted to produce one or more treated areas
5A—that are not damaged to a clinically significant degree,
5B—that are barely visible under normal room lighting,
5C—that have short persistences (typically lasting significantly only a few days post-treatment) and
5D—that produce little or no light scattering that affects corneal clarity.

6. The device of claim 5 that is used for combination therapy including laser light treatment plus one of more of the following therapies including, but not limited to: pharmacologic agents including, but not limited to, atropine; orthokeratology; and vision aid devices including, but not limited, to specialized spectacles and specialized multifocal contact lenses.

7. The device of claim 5 that is usable in multiple treatments that are adjusted to maintain sufficient hyperopia reserve to prevent 0.2 or more diopters per year of myopia increase.
